(19) United States
(12) Patent Application Publication
(10) Pub. No.: US 2010/0312216 A1
(43) Pub. Date: Dec. 9, 2010

(54) Potty training liners

(76) Inventor: Lori Lee Periman, Cedar Park, TX (US)

Correspondence Address: The Law Office of James E. Harris, P.O. BOX 872557, Vancouver, WA, 98687, US

(21) Appl. No.: 12/803,006

(22) Filed: Jun. 16, 2010

Related U.S. Application Data
(63) Continuation-in-part of application No. 12/285,586, filed on Oct. 9, 2008.
(60) Provisional application No. 60/998,825, filed on Oct. 12, 2007.

(51) Int. Cl. A61F 13/53; A61F 13/58

(52) U.S. Cl. 604/385.04; 604/385.101; 604/385.03

(57) ABSTRACT

Potty training liners are a comfortable, child sized, absorbent liner, with a concave center and an expandable, flexible bottom to allow rapid flowing urine collection and retention. The Liner has unique absorbent collapsible side walls with an unobstructed center containing superabsorbent for quick pooling of urine. The liner is adhered to the inside of the child's underwear with a thick adhesive strip on the bottom outer portion of the liner. The Liner is soft and flexible, and does not have the feel of a diaper. It is a unique stop-gap solution for children progressing from pull-on diapers to unprotected underwear. The purpose of the liner is for training, and short term emergency “accident” protection, as opposed to long term wearable protection.

### CROSS-REFERENCE TO RELATED APPLICATIONS

This application claims priority to and is a continuation-in-part of co-owned, co-pending U.S. patent application Ser. No. 12/285,586, filed Oct. 9, 2008, which claims priority to United Stated Provisional Patent Application Ser. No. 60/998,825, filed Oct. 12, 2007, each of which is incorporated herein by reference in its entirety.

### BACKGROUND

1. Field of the Invention

The present disclosure relates generally to devices that function as an aid to children transitioning from diapers to underwear.

2. Description of Related Art

Pull-on type diapers are presently used for potty training, as they allow a child to pull them down, like underwear, to go to the bathroom.

### BRIEF DESCRIPTION OF THE DRAWINGS

FIG. 1 contains a top view of a potty training liner embodiment adapted for use by a girl.

FIG. 2 contains a transverse partial cross-sectional view taken near the rear liner wall of the FIG. 1 embodiment.

FIG. 3 contains a top view of a potty training liner embodiment adapted for use by a boy.

FIG. 4 contains a top view of a potty training liner embodiment adapted for use overnight.

FIG. 5 contains a transverse cross-sectional view taken through an embodiment

FIG. 6 contains a top, side, and two cross-sectional views of another potty training liner embodiment.

FIG. 7 illustrates the FIG. 6 embodiment in perspective view.

FIG. 8 contains additional top, side, and perspective views of the FIG. 6 embodiments, flexed into an approximate “as-worn” configuration.

FIG. 9 contains a top, side, and two cross-sectional views of yet another potty training liner embodiment.

### DETAILED DESCRIPTION

Although pull-on type diapers do not keep children, who can sense that they need to go to the bathroom, from going, it is now believed that pull-on type diapers have characteristics that do not assist the child in progression from the security of a diaper to wearing unprotected underwear. Pull-on type diapers have a bulky “diaper” feel to them, like the traditional diapers a child has worn since birth. Accordingly, the use of pull-on type diapers for potty training can send confusing messages to children.

The present embodiments are designed for use with underwear, as a transition between traditional or pull-on type diapers and underwear alone. These embodiments are referred to herein as “potty training liners,” and consist of disposable inserts for use with underwear. Potty training liners are preferably designed to be used after a child gains some bladder control (e.g., using pull-on type diapers) and is ready to progress to wearing real underwear. Underwear outfitted with potty training liners does not have the “diaper-feel” of pull-on diapers, and yet gives some protection against unimpeded urine flow onto clothing, carpets, and furniture, etc.

Potty training liners enable a child to wear real underwear earlier while reducing the risk of unnecessary embarrassment and clean-up. Most children do not want to soil their clothes and are often embarrassed at their mistake. A child who has some bladder control knows he “has to go,” but simply waits too long, or gets distracted and forgets he has to go. This typically results in a child not quite making it to the bathroom in time. Potty training liners can save a child in this situation from soiling their clothes, and other items, and the bulky feel of a saturated liner creates awareness prompting the child to take action earlier and allowing them to learn.

Potty training liners are designed for preferable use as “short-term” emergency protection, whereas diapers and sanitary pads are designed for “long-term” wearable protection. At least some preferred embodiments of potty training liners are not designed to keep wetness away from the child, or to allow a child to continue playing after having an accident, but simply keep wetness confined to the pad until they can get to a bathroom or get assistance.

It is now believed that the use of potty training liners will speed the process of potty training by getting children into real underwear much faster, and creating greater awareness of the child's need to go, when they first realize they should, without the added negative consequences of having an unprotected accident. Many busy parents keep children in diapers or pull-on diapers longer, because they fear the clean-up and hassle. Often, parents do not have the time to watch their child as closely as is required when transitioning to real underwear, and frequently forget to remind their child to go to the bathroom. Additionally, liners are preferable to pull-on diapers because they are easier to change. While the pull-on diapers have easy-tear sides for quid(removal, the child must still fully undress to put on a clean dry pull-on diaper. Potty training liners will save time by cutting down on clothing changes and redressing. Ultimately, potty training liners help the child “self-train” and keep the experience positive. 191 Sanitary and incontinence pads currently exist in the marketplace for use by adults, but are not believed to meet the needs of a child learning bladder control. Perhaps most important, neither sanitary nor incontinence adult pads will fit or function properly for a child. Sanitary pads are developed to absorb slower, thicker body fluids and hold them over long periods of time. Likewise, incontinence pads are designed for incontinent adults, who typically produce small urinary leaks over time. Children in the potty training process typically have the bladder control to hold their urine until their bladder is nearly full, followed by an urgent release that produces a high volume of urine in a brief time period. Adult incontinence pads are far too large and bulky for a child. In addition, adult incontinence pads are not designed to aid the potty training process because they do not have a dry lightweight feel required for training and differentiating the bulky diaper-like feel between a child's legs.

The preferred embodiments do not protect the child from wetness, as this is not conducive to potty training, nor do they provide long-term wet wearability, like a diaper or other pad. The preferred embodiments do, however, contain several features designed to enhance the comfort and wearability of the dry liner. In preferred embodiments, training liners do not have the feel of a diaper or pull-on training-pant, which has the feel of a diaper. The size and shape, along with compressible side walls, give a comfortable, flexible fit for a small child while providing substantial protection from unexpected puddles on floors and furniture. The flexible bottom and absorbent side wall design can retain larger volumes of urine more quickly, by giving the urine somewhere to go besides over the sides, catching and containing it at a faster rate than existing pads. In some embodiments, the side wall design provides greater useable surface area for rapid urine absorption than a pull-on training pant, further enhancing the ability to prevent inadvertent spills. The liners in some embodiments have a form fitting rise at the middle, and rear, of the liner in attempt to protect overflow along the natural curves of the body, while urine is absorbed. The liners can also have adhesive limited to the outer portions of the underside of the liner, so that the center can flex and provide expansion as urine is collected and absorbed. This design works with cloth undergarments which naturally give a little in the center.

It is preferable to progress to training liners over pull-on diapers for several reasons. Currently, pull-on diapers require undressing and redressing to change into a new dry pull-on diaper, and they have the same feel as an infant's diaper. Further, pull-on diapers disperse urine in the pant, providing long term wear-ability. This has the effect of numbing awareness and encouraging a child to continue playing and not address the situation. Children must learn to take action to prevent accidents. Part of this learning process is knowing “when” to act, especially when attention is focused on something exciting, like playing. The preferred liners collect urine more centrally, increasing awareness. As the preferred liner becomes saturated, it feels awkward and heavy between the child's legs, prompting the child to take action. Furthermore, training liners do not require a full undress and redress to change into a new dry liner, making the change faster and easier for parents, and giving kids the ability to self-train.

Most absorbent products on the market are designed with at least three layers consisting of a liquid permeable top layer, an absorbent core, and a non-permeable bottom layer. Absorbent materials used in various absorbent products are known. Typically, such products comprise an absorbent fibrous matrix of cotton or wood pulp fluff enhanced with a high-absorbency material known as “superabsorbent.” Superabsorbents are typically crosslinked polymers capable of absorbing 10-100 times their weight in water. Superabsorbents are frequently used in diapers and personal care products to enhance absorption. There are many ways, known to those in the art, to combine superabsorbent and absorbent materials. For example, U.S. Pat. No. 5,356,403, issued to Faulks, et al., describes a layered fibrous structure laced with varying amounts of superabsorbent material at various densities. Other methods involve weaving the superabsorbent into a single fibrous pad during production, or sandwiching a superabsorbent between two fibrous layers.

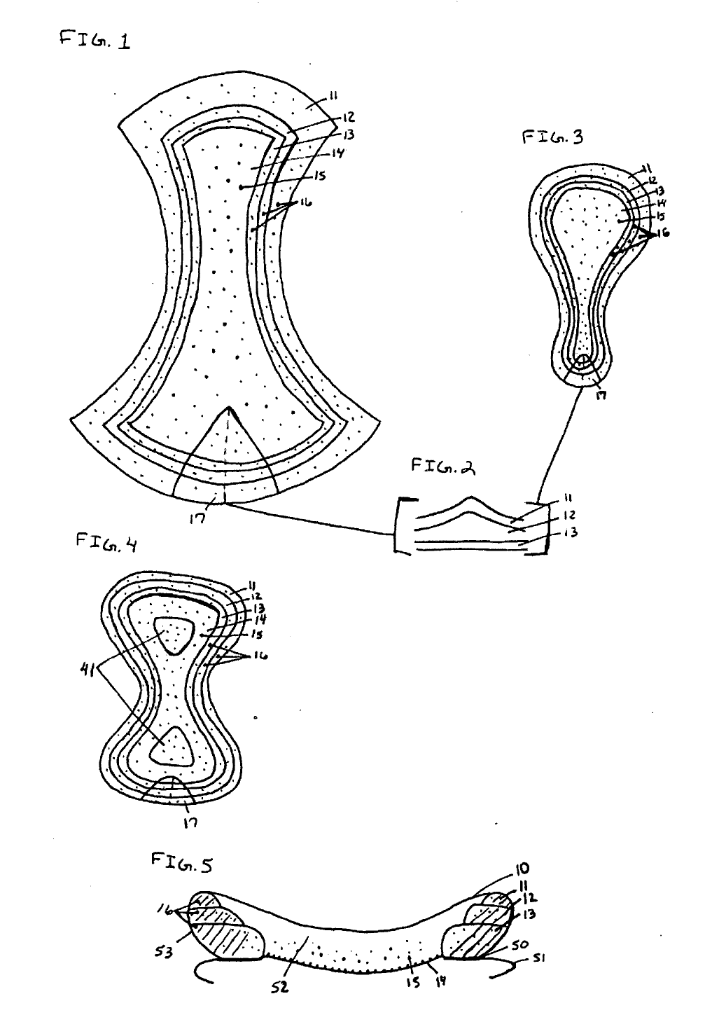

The preferred embodiments are illustrated in FIG. 1 (Top view of liner adapted for girls); FIG. 2 (partial rear transverse cross section); FIG. 3 (Top view of liner adapted for boys); FIG. 4 (Top view of liner adapted for use overnight); and, FIG. 5 (traverse cross-section). The first layer 10, (FIG. 5) comprises a thin, soft, absorbent top sheet, such as absorbent Rayon-polyester with low density, or other non-woven material, which covers and protects the underlayers (the top layer is removed in the drawings FIGS. 1, 2, 3, and 4). Urine moves immediately through this top layer with minimal resistance.

The bottom layer 14 is a soft flexible waterproof material which contains loose superabsorbent material, with or without sparse fibrous material mixed in to aid in preventing gel block. 15. The superabsorbent is typically a hydrophilic polymer made of fine particles of an acrylic acid derivative, such as sodium acrylate, potassium acrylate, or an alkyl acrylate. Further, the flexible bottom layer 14 has a light coating of adhesive on the inside to hold some of the powdered superabsorbent in place and to ensure coverage. The loose superabsorbent 15 allows gravity to dictate where it is needed most. The bottom 14 is designed to flex, this allows side compression for comfort and expansion for increased urine retention. Unlike current absorbent pads, cloth underwear gives and will allow additional expansion in the event of an accident.

The center of the liner 52 is substantially hollow, or concave, so that urine may flow easily into the space while allowing the top layer 10, and bottom layer 14, to sag when the sides are compressed creating space for urine to pool as it is being absorbed. Currently, when sanitary napkins or diapers are compressed on the side, the pad center has a tendency to bunch “up.” This creates overflow, when fluid hits the surface faster than it can be absorbed. Allowing the center to compress downward provides a more comfortable fit, and affords space for urine retention, while avoiding the stiff feel of a diaper or pad.

The center of the liner 52 is surrounded by a soft absorbent wall (11, 12, 13). The preferred wall configuration contains stacked absorbent and superabsorbent material in a stair-step fashion extending outward. The wall is made of three layers, a bottom layer 13, a middle layer 12 set off-center toward the outside, and a top layer 11 set off-center toward the outside. This creates a lower profile, collapsible wall, that minimizes blockage of urine flow to the center of the liner. Each layer contains superabsorbent 16 which causes the wall to swell, further trapping urine. This configuration allows for sides to compress comfortably while absorbing and directing fast streaming urine toward the center of the liner where it can be contained until the superabsorbent has had time to work. Extra absorbent material in the liner mid-section wall 11, 12, 13 can provide additional protection along the natural curve of the body.

The fibrous absorbent material used in the wall (11, 12, 13) may be formed from natural or synthetic fibers and by using methods such as air laying, spunbond, meltblown, or any of the methods known to those skilled in the art for making absorbent fibrous materials. The fibrous layer contains a superabsorbent 16. The superabsorbent may be dispersed through the fibrous layer at the time the fibrous layer is created, or sandwiched between fibrous layers, or by any means available to those skilled in the art.

Additionally, the top two layers in the wall of the rear area 17 may have extra absorbent material, creating a form-fitting rise to help catch potential overflow to the rear when sitting or lying down.

Liners may be designed as unisex, but for comfort, and a lower profile, the preferred liners will be designed based upon gender specific needs. The boy's liner, FIG. 3, will be a somewhat relaxed “sport-cup” shape, with a larger catch area in the front, and an extended reserve in the rear area.

Liners for Girls, FIG. 1, will be a more oblong hourglass shape, with a larger catch area in the rear, and a larger reserve area in the front, but will not extend as far in the front.

Liners designed for overnight protection, FIG. 4, will be longer, as well as contain extra absorbent material in the walls (11, 12, 13), and in the front and rear center 41 sections, to help catch urine while a child is lying down.

Liners designed for advanced trainers, who are almost finished with potty-training, will have thinner walls and a lower profile, to catch the little leaks that escape when they are trying to get to the bathroom.

Colors can vary, and may include fun child like designs, and/or a disappearing smiley, or positive reinforcement symbol on the inside top layer of the pad, as a positive indication that the pad is dry. (Ink could disappear, or change to “try again” or “oops” type symbol when wet.)

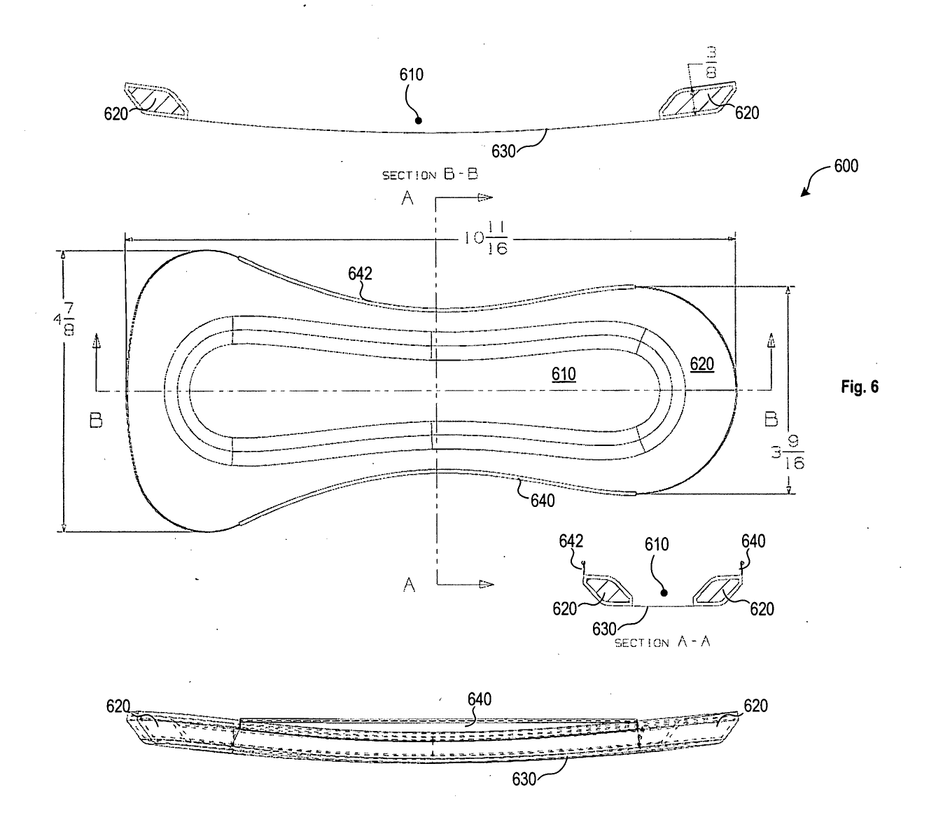

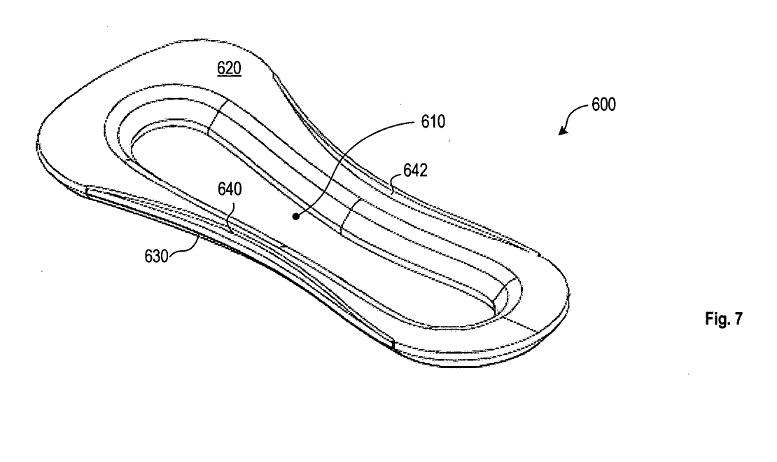

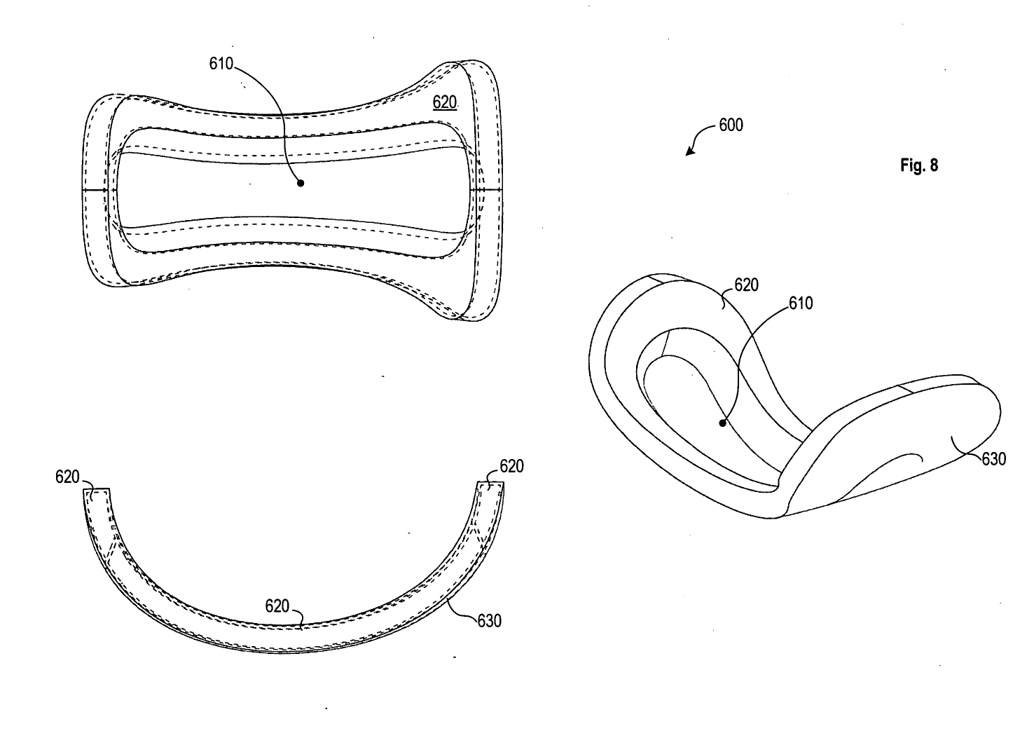

An alternative to a pad incorporating absorbent layers is a formed pad 600 such as shown in FIGS. 6, 7, and 8. Formed pad 600 is machine-shaped and pressed into a waterproof liner 630 to leave a central void 610 with the bulk of the absorbent material contained in a peripheral region 620. The peripheral region 620 can be filled with superabsorbent, pulp, fluff, and combinations thereof. In this embodiment, the cross-section of the peripheral sidewalls has an approximately parallelogram shape to aid comfort. A water-permeable top sheet placed over the top surfaces of the pad 600 and pressed to them, e.g., with adhesive, can help the pad hold its shape. An outer side edge 640, 642 forms side gathers to help prevent leakage. The side gathers can be elasticized to help form a deployed pad to the child's shape, as shown in FIG. 8. Adhesive means (not shown) are applied to the underside of the liner or on side wings to adhere the pad to a child's undergarment. Preferably, a central region of the pad does not contain adhesive, allowing the pad to move naturally and fill space left by the undergarment's flexing.

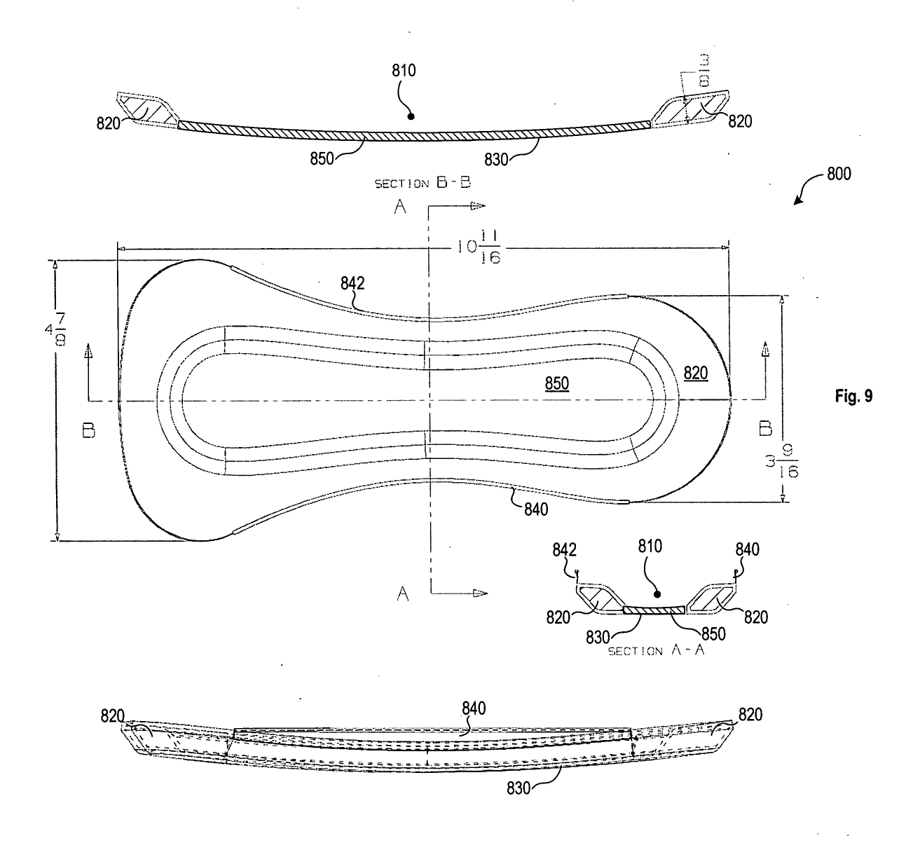

FIG. 9 shows an additional embodiment 800. Like embodiment 600, pad 800 has a central void 810, an absorbent peripheral region 820, a waterproof liner 830, and side gathers 840, 842. Instead of the top sheet being bonded directly to the waterproof liner in the central void region, however, pad 800 incorporated additional absorbent/superabsorbent material in a central absorption region 850, which underlies central void 810. Central absorption region can contain the same materials as the peripheral absorption region, or can use strictly superabsorbent materials if the peripheral region contains pulp or fluff. Preferably, the central absorption region is relatively thin if employed, so that the center of the pad remains easily deformable and contains a large void region. The central absorption region 850 assists the peripheral absorption region 820 in absorbing fluid that is quickly pooled into the central void. Preferably, however, most of the absorbing capacity (e.g., 75% or more) remains in the peripheral region so as not to unduly reduce the size of the central void.

The central void need not be completely bereft of material in a given embodiment. It can be filled, completely or partially, with an extremely low-density material that has relatively little permanent fluid-holding capacity, as compared to the absorption regions of the pad, but low impedance to fluid flow when saturated. Such a low-density material could help the pad maintain its shape and provide additional wet, unpleasant feedback to a child having an accident.

The absorbent sections of the liner can be formed, e.g., using known methods and materials from the art. For instance, pulp/fluff material formed in a drum can be pressed to the desired shape of the absorbent portions of the liner. A superabsorbent material can be used instead, and/or the superabsorbent material can be mixed with pulp/fluff. One example of a superabsorbent material is Infinicel™, made by Proctor and Gamble, Inc. An alternative is the silver-containing absorbent material marketed as Nanosan®.

Although several embodiments and alternative implementations have been described, many other modifications and implementation techniques will be apparent to those skilled in the art upon reading this disclosure. Materials described as water-permeable may incorporate water-resistant components, but with an overall permeability. Likewise, materials described as waterproof may be water-resistant, and additionally allow some water to pass with extracted exposure times. Dimensions shown on Figures are suggested for one size embodiment, with other dimensions appropriate for other applications.

Although the specification may refer to “an”, “one”, “another”, or “some” embodiment(s) in several locations, this does not necessarily mean that each such reference is to the same embodiment(s), or that the feature only applies to a single embodiment.

FIG. 1. Top Down View of Girl's Liner with Top Sheet Removed:
11. Top layer of absorbent outer wall containing superabsorbent
12. Second layer of absorbent outer wall containing superabsorbent
13. Third layer of absorbent outer wall containing superabsorbent
14. Flexible waterproof bottom layer, coated with light dusting of adhesive and superabsorbent
15. Loose superabsorbent, or loose superabsorbent mixture with a fibrous material
16. Superabsorbent disbursed through side walls
17. Absorbent material added to rear wall to form an inverted V for added leak protection

FIG. 2. Exploded Horizontal View of the Rear Liner Wall:
11. Rear overflow protection. Top layer of absorbent wall with additional absorbent material forming a protective peak in rear
12. Rear overflow protection. Second layer of absorbent wall with additional absorbent material forming a protective peak in rear
13. Third layer of absorbent wall with superabsorbent material

FIG. 3. Top Down View of Overnight Liner with Top Sheet Removed:
11. Top layer of absorbent outer wall containing superabsorbent
12. Second layer of absorbent outer wall containing superabsorbent
13. Third layer of absorbent outer wall containing superabsorbent
14. Flexible waterproof bottom layer, coated with light dusting of adhesive and superabsorbent
15. Loose superabsorbent, or loose superabsorbent mixture with a fibrous material
16. Superabsorbent disbursed through side walls
17. Absorbent material added to rear wall to form an inverted V for added leak protection

FIG. 4. Top Down View of Overnight Liner with Top Sheet Removed:
11. Top layer of absorbent outer wall containing superabsorbent
12. Second layer of absorbent outer wall containing superabsorbent
13. Third layer of absorbent outer wall containing superabsorbent
14. Flexible waterproof bottom layer, coated with light dusting of adhesive and superabsorbent
15. Loose superabsorbent, or loose superabsorbent mixture with a fibrous material
16. Superabsorbent disbursed through side walls
17. Absorbent material added to rear wall to form an inverted V for added leak protection
41. Extra absorbent material containing superabsorbent added to front and rear center

FIG. 5. Horizontal End Cut Away View:
10. Low density, liquid permeable top sheet
11. Top layer of absorbent outer wall containing superabsorbent
12. Second layer of absorbent outer wall containing superabsorbent
13. Third layer of absorbent outer wall containing superabsorbent
14. Flexible waterproof liner bottom, coated inside with adhesive and superabsorbent
15. Free floating superabsorbent, or superabsorbent mixture with a fibrous material
16. Superabsorbent located inside wall material
50. Adhesive applied to bottom of flexible liner, under wall, to attach liner to underwear
51. Adhesive flap to wrap around underwear seam and secure liner to underwear
52. Substantially hollow, concave center

What is claimed is:

1. An elongated absorbent pad comprising:
a substantially waterproof shell layer;
a peripheral absorption region lying substantially above outboard regions of the shell layer; and
a central fluid distribution region above the shell layer and enclosed by the peripheral absorption region, the central fluid distribution region creating a pooling area for liquid to be absorbed by the peripheral absorption region.

2. The elongated absorbent pad of claim 1, wherein the peripheral absorption region contains at least 75% of the total absorptive capacity of the pad.

3. The elongated absorbent pad of claim 1, wherein the pooling area created by the central fluid distribution region has sufficient volume to temporarily retain at least half of the total absorptive capacity of the peripheral absorption region.

4. The elongated absorbent pad of claim 3, wherein the central fluid distribution region comprises a region of relatively low-density, rapid takeup absorbent material, as compared to the peripheral absorption region.

5. The elongated absorbent pad of claim 1, wherein the central fluid distribution region comprises a depressed void with lateral extent defined by sidewalls of the peripheral absorption region.

6. The elongated absorbent pad of claim 5, wherein a central absorption region underlies the depressed void, in an unused pad the central absorption region occupying less volume than the depressed void.

7. The elongated absorbent pad of claim 6, further comprising a top water-permeable layer overlying the peripheral absorption region and the central absorption region.

8. The elongated absorbent pad of claim 7, the central absorbent region comprising a superabsorbent material dispersed between the shell layer and the top water-permeable layer.

9. The elongated absorbent pad of claim 8, further comprising an adhesive layer on the shell layer, at least some of the superabsorbent material adhering to the adhesive layer.

10. The elongated absorbent pad of claim 7, wherein the top water-permeable layer further comprises a positive reinforcement mark, which is visible when the absorbent pad is dry and fades when the absorbent pad is wet.

11. The elongated absorbent pad of claim 1, wherein the peripheral absorption region comprises a superabsorbent material.

12. The elongated absorbent pad of claim 1, wherein the peripheral absorption region comprises a plurality of vertically overlapping absorbent layers, the upper one of the overlapping absorbent layers having an inner periphery larger than the lowest one of the overlapping absorbent layers.

13. The elongated absorbent pad of claim 1, the peripheral absorption region abutted by a waterproof outer edge connected to the non-water-permeable shell layers.

14. The elongated absorbent pad of claim 1, the peripheral absorption region comprising, in the rear center of the peripheral absorption region, a thicker peaked absorbent section.

15. The elongated absorbent pad of claim 1, wherein the shell layer comprises an expandable material.

15. The elongated absorbent pad of claim 1, further comprising an adhesive layer applied to the outside surface of the shell layer.

16. The elongated absorbent pad of claim 15, wherein the adhesive layer is limited to side regions of the shell layer.

17. The elongated absorbent pad of claim 1, further comprising an adhesive side flap connected to the shell layer, the adhesive side flap having adhesive on its underside.

18. The elongated absorbent pad of claim 1, the central fluid distribution region having an enlarged front section adapted to cup a male child's genitals within the peripheral absorption region.

19. A method of containing body fluid within an absorbent pad, the method comprising:
temporarily holding and distributing the body fluid within a central distribution region of the pad; and
absorbing at least most of the body fluid into a peripheral absorption region of the pad surrounding the lateral sides of the central distribution region.

20. The method of claim 19, further comprising absorbing a minority portion of the body fluid in a central absorption region underlying the central distribution region.

21. The method of claim 19, further comprising attaching the pad to a child's undergarment such that the central distribution region is positioned to catch urine released by the child.
